(19) United States
(12) Patent Application Publication
(10) Pub. No.: US 2016/0369229 A1
(43) Pub. Date: Dec. 22, 2016

(54) MATERIAL FOR CELL CULTIVATION, PRODUCTION METHODS AND USES THEREOF

(71) Applicant: UNIVERSITE DE ROUEN, Mont-saint-aignan (FR)

(72) Inventors: Elias AL TAWIL, ROUEN (FR); Alexandre MONNIER, BAILLEUL-NEUVILLE (FR); Quang Trong NGUYEN, LE HAVRE (FR); Stephane MARAIS, MALAUNAY (FR); Yusra KASSIM-SABRI, ROUEN (FR); Elise DEMANGE, MONT SAINT AIGNAN (FR); Brigitte DESCHREVEL, MONT SAINT AIGNAN (FR)

(21) Appl. No.: 15/120,851

(22) PCT Filed: Feb. 24, 2015
(86) PCT No.: PCT/FR2015/050441

§ 371 Date: Aug. 23, 2016

(30) Foreign Application Priority Data
Feb. 24, 2014 (FR) 14 51478

(51) Int. Cl. C12N 5/00; C12N 5/0775; C12N 5/071

(52) U.S. Cl. CPC C12N 5/0068; C12N 5/069; C12N 5/0668; C12N 2533/40; C12N 2535/00; C12N 2533/00; C12N 2533/30; C12N 2502/30

(57) ABSTRACT

A double-porosity three-dimensional material includes a support consisting of a polymer having a surface which includes positive charges and is functionalized by bioactive molecules which include negative charges and are used to increase the proliferation of eukaryotic cells. A method of producing the double-porosity three-dimensional material, and use of the material for fixing and stimulating the proliferation of eukaryotic cells are also described.

### TECHNICAL FIELD

The present invention relates to double-porosity materials comprising a support constituted of a polymer of which the surface is functionalized with bioactive molecules, and also to the processes for preparing same and to the uses thereof for attaching and proliferating eukaryotic cells.

### BACKGROUND OF THE INVENTION

Over the past twenty years, various types of supports, allowing the culture of eukaryotic cells, have been gradually developed with the aim of creating devices intended essentially for the study of cell behavior and for tissue engineering.

Supports of hydrogel type are, for example, known. However, hydrogels derived from molecules which are compatible with the development of cells in vivo have a low availability. Furthermore, most hydrogels are either disaggregated by body fluids, or incapable of maintaining good mechanical properties for the period of time required for tissue regeneration.

Supports of ceramic porous type are also known. However, porous ceramics are very rigid and are difficult to shape.

The use of biopolymers such as collagen, fibrin, alginate or chitosan is also described for producing supports intended for the culture of eukaryotic cells. These biopolymers have the advantage of possessing good biological properties, and of being biocompatible and biodegradable. However, while their physical surface properties are advantageous, their overall mechanical properties are generally weak, even after chemical crosslinking, thereby limiting their field of application.

The design of suitable supports must meet many requirements which can, however, be modulated according to the applications envisioned. The support must, first and foremost, allow the cells to adhere to its surface. It must also allow cell proliferation and survival, which implies that the cells can, on the one hand, gain access to nutrients and to various growth factors and, on the other hand, eliminate waste. Also, according to the cell types considered, the support must also allow cells to change shape, to migrate, or even to differentiate. The support must consequently have appropriate chemical and physical properties. From the point of view of the chemical properties, the support must first and foremost be biocompatible and nonimmunogenic. According to the application envisioned, the support may advantageously be biodegradable. Its rate of biodegradation must then be suitable for the intended application and the products resulting from this process must be nontoxic. In addition, the chemical composition of the surface of the support must allow cell adhesion. The physical properties of the support may relate to the support as a whole, for instance the rigidity of a bone prosthesis or the flexibility of a capillary or a suture thread. They also and specifically relate to the surface of the support. Indeed, the roughness and also the porosity of the support can play a determining role in the adhesion and proliferation of the cells. The mechanical properties of the surface of the support, for their part, play a role with respect to the ability of cells to change morphology and to migrate. Added to these chemical and physical properties are other criteria such as the ease of preparation of the support, the possibilities of shaping said support, the possibility of sterilizing it, the ease with which it can be used or its cost. Advantageously, the support may be prepared with biobased materials and may be produced on a large scale.

In this context, the inventors have developed a polymer support with double porosity, an essential feature for allowing cells to organize in the form of tissues and for enabling the vascularization and optionally the resorption of the implant. The internal and external surfaces of this support are totally functionalized with biomolecules by means of a simple and mild technique which preserves the structure and the functions of the biomolecules.

### SUMMARY OF THE INVENTION

An object of the present invention is a three-dimensional material with double-porosity comprising a support constituted of a polymer of which the surface comprises positive charges and is functionalized with bioactive molecules which comprise negative charges and are suitable for increasing eukaryotic cell proliferation.

An object of the present invention is also a process for producing a three-dimensional material, comprising the following successive steps:

1—forming a polymer by phase inversion so as to obtain a two- or three-dimensional support with double-porosity,
2—treating the surface of the support so as to confer positive charges thereon,
3—functionalizing the thus-treated support with bioactive molecules.

Another object of the invention is the use of the material according to the present invention for attaching and stimulating the proliferation of eukaryotic cells.

### DETAILED DESCRIPTION OF EMBODIMENTS

Three-Dimensional Material with Double-Porosity

The material object of the present invention comprises a polymer support. The surface of said support is treated so as to introduce positive charges onto said support. Bioactive molecules comprising negative charges are immobilized on the surface of said support by electrostatic interactions with the positive charges. The bioactive molecules are in particular suitable for increasing eukaryotic cell proliferation. Said molecules are able to interact with eukaryotic cells, and are, for example, molecules of tissue extracellular matrix. The bioactive molecules are in particular present on the internal surface, i.e. in the macropores and the micropores, and on the external surface of the support.

The material object of the present invention differs from materials comprising pieces of polymer linked to one another by a bioactive-molecule gel crosslinked by a cationic crosslinking agent. Indeed, the support of the material according to the invention is in the form of a single piece. Furthermore, in the material according to the invention, the bioactive molecules are immobilized on the support by electrostatic interactions and are not crosslinked with one another, i.e. they can fan out freely, without constraints due to chemical bonds. Thus, in the material of the present invention, the bioactive molecules organize at the surface of the support in the form of fibers. Said fibers are more or less dense and more or less entangled depending on the amount of negative charges that they carry and the electrostatic repulsions between the chains of bioactive molecules.

According to one particular embodiment, the polymer support of the material according to the invention is a bioresorbable polymer support.

According to another particular embodiment, the polymer support of the material according to the invention is a non-bioresorbable polymer support.

The polymer of the support can in particular be a polyester or a mixture thereof.

Examples of non-bioresorbable polyester that can be used to form the support are polymethacrylate, polyacrylate, poly(ethylene terephthalate), and mixtures thereof.

Examples of bioresorbable polyester that can be used to form the support are homopolymers and copolymers of hydroxy acids, such as polylactic acid (PLA), polyglycolic acid (PGA), lactic and glycolic acid copolymers (PLGA), poly(3-hydroxybutyrate), poly(4-hydroxybutyrate), poly(3-hydroxyvalerate), poly(5-hydroxyvalerate), poly(3-hydroxypropionate), poly(3-hydroxyhexanoate), poly(3-hydroxyoctanoate), poly(3-hydroxyoctodecanoate); polycaprolactone; homopolymers and copolymers of poly(butylene succinate) and of poly(butylene adipate); and mixtures thereof.

According to one particular embodiment, the polymer is a bioresorbable polyester, more particularly the polymer is polylactic acid (PLA). This synthetic polymer is known for its biocompatibility and its biodegradability. According to one particularly preferred embodiment, the polyester is a PLA comprising at least 95% of L enantiomer and at most 5% of D enantiomers. Indeed, poly(L-lactic acid) is degraded in vivo to L-lactic acid, a species that is naturally metabolized by the human body. Recognized and approved from this point of view by most health authorities, in particular by the “Food and Drug Administration” in the United States, PLA is currently used for the production of various implants and suture threads. Furthermore, it can be subjected to various types of forming.

The polymer of the support of the material of the present invention can in particular have a weight-average molar mass (M_{w}) of from 100,000 to 800,000 g·mol⁻¹, in particular from 140,000 to 750,000 g·mol⁻¹.

The polymer of the support of the material of the present invention can in particular have a number-average molar mass (Mₙ) of from 50,000 to 400,000 g·mol⁻¹, in particular from 55,000 to 300,000 g·mol⁻¹.

The bioactive molecules immobilized on the support can in particular be chosen from polysaccharides such as hyaluronan, chondroitin sulfates, chitosan and heparin; proteins such as fibrinogen and collagen, in particular soluble collagen; peptides such as those known for their cell adhesion capacity, for instance those containing the arginine-glycine-aspartic acid (RGD) or arginine-glycine-aspartic acid-serine (RGDS) sequence; and mixtures thereof. Gelatin is not a bioactive molecule for the purposes of the present invention since it does not interact with eukaryotic cells.

According to one preferred embodiment, the bioactive molecules are hyaluronan or hyaluronic acid (HA) molecules. Indeed, HA is a major constituent of the extracellular matrix of human tissues. It is a glycosaminoglycan of high molar mass (1×10⁶ to 10×10⁶ g·mol⁻¹), constituted of repetitive D-glucuronic acid-β(1,3)-N-acetyl-D-glucosamine disaccharide units linked to one another by β(1,4) glycosidic bonds. By virtue of its unique biophysicochemical properties (viscoelasticity, high water retention capacity, capacity to interact specifically or nonspecifically with various proteins), HA plays a fundamental role in extracellular matrix organization and homeostasis. Also, it is established that the physicochemical properties and biological functions of HA are strongly dependent on its chain size. For example, while HA of high molar mass is anti-angiogenic and inhibits inflammation, HA oligosaccharides stimulate angiogenesis and activate the innate immune response. Thus, it is possible to modulate the properties of the supports by adjusting the size of the HA chains used for the functionalization thereof.

The material object of the present invention has a double porosity, i.e. it has a network of pores of two different sizes. The material can in particular comprise interconnected micropores and macropores. The micropores can in particular have a diameter of less than 20 μm, in particular from 0.1 to 10 μm, more particularly from 1 to 5 μm. The micropores allow rapid transport of nutrients and gases to the cells which proliferate on the support, and also the evacuation of waste. The macropores can in particular have a diameter of from 20 to 199 μm, in particular from 40 to 190 μm, more particularly from 50 to 180 μm. The macropores receive the cells allowing them to proliferate on a large internal surface area of the support while at the same time allowing their recovery in the living state.

For the purposes of the present invention, the term “pore diameter” is intended to mean the average diameter of the pores as measured according to the method described herein.

According to one particular embodiment, the material has regular macropores which open onto one face of the support and interconnected micropores located between the macropores.

The support of the material object of the present invention can in particular have a void fraction of from 60% to 85%, in particular from 65% to 80%, more particularly from 70% to 75%.

For the purposes of the present invention, the term “void fraction” is intended to mean the percentage of the volume of the pores relative to the volume of the material, as measured according to the method described herein.

According to one particular embodiment, the polymer support does not have a nanofibrous structure.

The support of the material object of the present invention can in particular have a solid, hollow or microspherical shape. According to one particular embodiment, the support has a solid shape.

The expression “the support has a solid shape” or “solid support” is intended to mean, for the purposes of the present invention, that the support has the shape of a solid body, i.e. a three-dimensional geometrical figure delimited by flat or curved surfaces, such as in particular a polyhedron or a round body. Preferably, the solid support is a flat support, such as in particular a disk.

The expression “the support has a hollow shape” or “hollow support” is intended to mean, for the purposes of the present invention, that the support has the shape of a hollow body which is open at at least two ends, such as in particular a tubular support or a tube. The term “tubular” does not necessarily mean that the internal channel has a circular cross section. Indeed, all shapes can be envisioned for the cross section of the tube, such as square, rectangular, oval, etc. Furthermore, the shape and/or the surface area of the cross section of the tube are not necessarily uniform throughout the length of the tube. Furthermore, the tube may comprise one or more bends.

The expression “the support has a microspherical shape” or “microspherical support” is intended to mean, for the purposes of the present invention, that the support has the shape of a sphere having an average diameter of less than 1 mm.

When the support has a solid or hollow shape, the material may in particular be a porous asymmetrical membrane, i.e. the porosity of each face of the membrane is different. Thus, one of the faces of the membrane may have micropores that can stop all cell and viral species while at the same time being permeable to water, to dissolved salts, to molecular organic solutes and to macromolecules.

According to one particular embodiment, when the support of the material of the invention has a solid or hollow shape, the micropores have a diameter of less than 15 μm, in particular from 0.1 to 10 μm, more particularly from 1 to 5 μm, and the macropores have a diameter of from 90 to 199 μm, in particular from 100 to 180 μm, more particularly from 110 to 170 μm.

When the support has a microspherical shape, the material may in particular be a microbead having an average diameter of from 50 to 300 μm, preferably from 100 to 200 μm.

According to one particular embodiment, when the support of the material of the invention has a microspherical shape, the micropores have a diameter of less than 6 μm, in particular from 0.1 to 2 μm, more particularly from 0.5 to 1 μm, and the macropores have a diameter of from 20 to 50 μm, in particular from 25 to 45 μm, more particularly from 30 to 35 μm.

### Process for Producing the Material

The material object of the present invention can in particular be obtained by means of a process comprising the following successive steps:

1—forming a polymer by phase inversion so as to obtain a two- or three-dimensional support with double-porosity,
2—treating the surface of the support so as to confer positive charges thereon,
3—functionalizing the thus-treated support with bioactive molecules.

The polymer and the bioactive molecules which are part of the process which is the subject of the invention can in particular be as defined above.

### 1) Forming the Polymer by Phase Inversion

The step of forming the polymer by phase inversion can in particular comprise the steps of:
preparing a solution of the polymer comprising said polymer, at least one solvent for the polymer and optionally at least one pore-forming agent and/or at least one organic or inorganic compound;
introducing said solution of the polymer into an aqueous solution optionally comprising at least one other water-miscible solvent and/or at least one surfactant.

The step of forming the polymer by phase inversion can in particular make it possible to obtain a polymer support having interconnected micropores and macropores as defined above.

The forming step is carried out differently depending on whether the support has a solid, hollow or microspherical shape.

a) Forming a Solid or Hollow Support

According to one particular embodiment allowing a solid or hollow support to be obtained, said solution of the polymer comprises the polymer, at least one solvent for the polymer which is water-miscible, optionally at least one pore-forming agent and optionally at least one organic or inorganic compound. Said solution of the polymer is deposited on a solid or hollow substrate, and the phase inversion is carried out by immersion of said substrate in an aqueous solution comprising optionally at least one other water-miscible solvent.

The water-miscible solvent for the polymer may in particular be N,N-dimethylformamide, N-methylpyrrolidone, dimethylacetamide, dimethyl sulfoxide, and mixtures thereof.

Advantageously, said solution of the polymer is viscous so as to facilitate the depositing thereof on the substrate and to form a layer having a controlled thickness. Thus, the concentration of the polymer in the solution of the polymer can in particular be from 4% to 25% by weight, preferably from 6% to 12% by weight, relative to the weight of the solution of the polymer.

The addition of at least one pore-forming agent to the solution of the polymer advantageously makes it possible to modify the pore size in a controlled manner. Examples of pore-forming agents that can be used according to the present invention are poly(ethylene glycol) and polyvinylpyrrolidone. According to one particular embodiment, the pore-forming agent has a weight-average molar mass (M_{w}) of from 600 to 500,000 g·mol⁻¹, in particular from 5,000 to 50,000 g·mol⁻¹, more particularly from 10,000 to 20,000 g·mol⁻¹. According to one particular embodiment, the pore-forming agent is a poly(ethylene glycol) having a weight-average molar mass of from 5,000 to 10,000 g·mol⁻¹. According to another particular embodiment, the pore-forming agent is a polyvinylpyrrolidone having a weight-average molar mass of from 20,000 to 50,000 g·mol⁻¹.

The concentration of pore-forming agent in said solution of the polymer may in particular be from 10% to 100%, in particular from 20% to 80%, by weight relative to the weight of the polymer.

The solid or hollow substrate on which said solution of the polymer is deposited can in particular be made of glass, metal or plastic resistant to solvents, such as polytetrafluoroethylene (Teflon®), nylon 6,6 or poly(ethylene terephthalate).

Since the solvent for the polymer is water-miscible, a polymer film is gradually formed by coagulation when said substrate covered with polymer is immersed in an aqueous solution. The solid or hollow support thus takes the shape of the substrate on which it is deposited. Since the coagulation rate is different at the surface in contact with the water and at the surface in contact with the substrate, the formation of pores of different sizes is observed. Thus, the pores of the surface in contact with the substrate have a larger diameter than the pores of the surface in contact with the water.

The substrate covered with polymer film can advantageously be immersed in several successive aqueous solutions.

Once the solid or hollow support is obtained, said support is washed so as to remove all traces of the solvent for the polymer.

b) Forming a Microspherical Support

According to another particular embodiment which makes it possible to obtain a microspherical support, said solution of the polymer comprises the polymer, at least one volatile solvent with low water-miscibility and optionally at least one organic or inorganic compound. The phase inversion is carried out by introducing said solution of the polymer into an aqueous solution comprising at least one surfactant, followed by evaporation of said volatile solvent.

Examples of volatile solvents with low water-miscibility are ethyl acetate, dichloromethane and mixtures thereof. According to one preferred embodiment, the bioresorbable polymer is in solution in a mixture of volatile solvents comprising a good solvent for the polymer, such as dichloromethane or ethyl acetate, and one or more poor solvents for the polymer, such as propan-1-ol, butan-1-ol, hexane and mixtures thereof.

An example of a surfactant that can be used according to the present invention is polyvinyl alcohol.

The concentration of surfactant in the aqueous solution can in particular be from 1 to 30 g·l⁻¹, in particular from 5 to 15 g·l⁻¹.

The aqueous solution comprising at least one surfactant is advantageously continuously stirred in a controlled manner as soon as the solution of the polymer is introduced and up to complete evaporation of the volatile solvent.

Once the volatile solvent is evaporated, the solution containing the microspherical supports is filtered and the supports are washed with water in order to remove the surfactant.

The diameter of the microspherical supports is controlled by the stirring speed and the concentration of surfactant.

During the forming step resulting in a solid, hollow or microspherical support, if the solution of the polymer subjected to the phase inversion process comprises at least one organic or inorganic compound, said compound is incorporated in the matrix of the material, in particular in the form of fine particles. Examples of organic compounds that can be introduced into the solution of the polymer are pharmaceutical active agents, such as in particular antibiotics, anti-inflammatories, and mixtures thereof. Examples of inorganic compounds that can be introduced into the solution of the polymer are calcium phosphate ceramics, such as in particular tricalcium phosphates, hydroxyapatite, and mixtures thereof.

Once the support has been obtained and washed with water, it can optionally be stored before being introduced into the second step of the process. The support can in particular be stored in the dry state or else in water optionally comprising an asepticizing agent.

The supports obtained following the first step of the process object of the invention can in particular have a double porosity as defined above.

### 2) Treating the Surface of the Support

Once the support has been formed, the second step of the process object of the invention consists in treating the surface of the support so as to confer positive charges thereon.

According to one preferred embodiment, the surface treatment is carried out by aminolysis reaction of the polymer using a solution of at least one aliphatic α,ω-diamine. The aminolysis reaction can in particular be carried out by immersing the polymer substrate in a solution of at least one aliphatic α,ω-diamine, followed by rinsing, for example with ultrapure water.

According to one particular embodiment, the aliphatic α,ω-diamine is chosen from 1,6-hexanediamine, 1,2-ethanediamine, 1,8-octanediamine, and mixtures thereof. Preferably, the aliphatic α,ω-diamine is 1,6-hexanediamine.

During the aminolysis reaction, ester bonds of the support are broken and form amide bonds with one of the two amine functions of the aliphatic α,ω-diamine. The second amine function remains free in the form of a quaternary ammonium. The support thus comprises positive charges at its surface in a wide pH range.

The aliphatic α,ω-diamine can in particular be in solution in a solvent chosen from alcohols, water and mixtures thereof, in particular propan-1-ol or water, preferably water. Indeed, when the aminolysis reaction is carried out in water, more free amine functions are obtained on the surface of the support and the surface of the support is rougher.

According to one preferred embodiment, the concentration of the aliphatic α,ω-diamine in the solution is from 1 to 300 g·l⁻¹, preferably from 2 to 60 g·l⁻¹.

According to another preferred embodiment, the aminolysis reaction is carried out at a temperature of less than 30° C., preferably less than 25° C., more preferentially at a temperature close to 22° C. This in fact makes it possible to avoid degradation of the support.

The aminolysis reaction can in particular be carried out for a period of from 5 to 90 minutes, preferably from 10 to 60 minutes.

3) Functionalizing the Support

Once the surface of the support has been treated so as to be positively charged, the third step of the process object of the invention consists in functionalizing the support with bioactive molecules.

The functionalizing step of the process object of the invention can in particular be carried out by immersing the support in a solution of bioactive molecules, followed by rinsing, for example with ultrapure water.

The duration of the immersing of the support in the solution of bioactive molecules can in particular be from 1 to 120 minutes, preferably from 10 to 20 minutes.

The concentration of bioactive molecules in the solution in which the support is immersed can in particular be from 0.1 to 20 g·l⁻, preferably from 1 to 5 g·l⁻¹.

The bioactive molecules exhibit negative charges in their structure when they are in solution.

According to one particularly preferred embodiment, the bioactive molecules are HA molecules.

The HA can in particular have a weight-average molar mass of from 20,000 to 10,000,000 g·mol⁻¹. Such HA molecules can be obtained by degradation of HA of high molar mass using reactive oxygen species such as hydrogen peroxide.

Alternatively, the HA can have a weight-average molar mass of from 800 to 20,000 g·mol⁻¹. Such HA molecules can be obtained by enzymatic hydrolysis of HA of high molar mass, for example in the presence of hyaluronidase.

The pH of the solution of bioactive molecules in which the support is immersed can in particular be from 1 to 9.

When the bioactive molecules are polysaccharides, the pH of the solution of bioactive molecules varies according to the weight-average molar mass and the pKa of the bioactive molecules. When the bioactive molecules have a weight-average molar mass of greater than 20,000 g·mol⁻¹, the pH of the solution of bioactive molecules is preferably less than the pKa of the bioactive molecules, more preferentially less than pKa−0.5. When the bioactive molecules have a weight-average molar mass of less than 20,000 g·mol⁻¹, the pH of the solution of bioactive molecules is preferably greater than the pKa of the bioactive molecules, more preferentially greater than pKa+2.

When the bioactive molecules are HA molecules having a weight-average molar mass of from 20 000 to 10,000,000 g·mol⁻¹, the pH of the HA solution is preferentially from 1 to 2.9, preferably from 1.5 to 2.5. indice, the choice of a pH lower than the pKa of the HA limits the number of negative charges on the HA molecule and therefore the electrostatic repulsion of the long chains of HA with respect to one another.

When the bioactive molecules are HA molecules having a weight-average molar mass of between 800 and 20,000 g·mol⁻¹, the pH of the HA solution is preferentially from 4 to 8, preferably from 5 to 7. Indeed, the problem of repulsion of HA chains with respect to one another is no longer a problem for HA molecules of low molar mass. Thus, the choice of a pH much higher than the pKa of the HA maximizes the number of negative charges on the HA molecule and therefore the electrostatic interactions with the positive charges of the support.

When the bioactive molecule is a protein or a peptide, the pH of the solution of bioactive molecules depends on the pI (isoelectric point) of the bioactive molecule. The pH of the solution of bioactive molecules is preferentially from 2 to 11, more preferentially it is greater than pI−2.

According to one particular embodiment, two successive functionalization steps can be carried out with different bioactive molecules. For example, when the support is solid or hollow and when it has macropores on one of its faces and micropores on the other face, it is possible to functionalize one face with bioactive molecules of one type and the other face with other bioactive molecules. The face to be functionalized is brought into contact with the solution containing the bioactive molecule, the other face being masked by isolating it from the solution by means of a seal on the periphery.

### Use of the Material

The material object of the present invention can be used to attach and stimulate the proliferation of eukaryotic cells.

According to one particular embodiment of the invention, the eukaryotic cells that can be attached on the material are chosen from stem cells, fibroblasts, endothelial cells, cancer cells, and mixtures thereof. For example, the stem cells can be mesenchymal stem cells. The mesenchymal stem cells can in particular differentiate into chondrocytes in the presence of TGFbeta-3 growth factor. The endothelial cells can be human vascular endothelial cells (HMEC-1). The cancer cells can be colon cancer epithelial cells (CaCo-2, HT-29 and DLD1 lines). The eukaryotic cells are placed in suspension in an appropriate culture medium.

The material according to the present invention can advantageously be sterilized without degrading its properties, in particular by immersion in ethanol or in a mixture of water and ethanol for a few minutes or by gamma-irradiation according to the standards in force (EN ISO 11137-1:2006; EN ISO 9001:2008; EN ISO 13485:2012).

After the sterilization, the material is immersed in the suspension of eukaryotic cells and incubated at 37° C. in a controlled atmosphere.

The eukaryotic cells which are attached to the support and which proliferate thereon can advantageously migrate and establish cell-cell interactions in a co-culture.

The supports seeded with eukaryotic cells can then be used in vitro, ex-vivo or in vivo.

Examples of use of the material with eukaryotic cells in vitro are in particular:
studying cell behavior and interactions between the cells and their environment;
developing and testing active ingredients such as medicaments or cosmetic products,
developing and testing therapeutic tools such as radiation;
toxicological studies for allergens or pollutants;
molecular screening.

Examples of use of the material with eukaryotic cells ex vivo, i.e. eukaryotic cells taken from a patient, are in particular:
carrying out diagnoses;
testing the efficacy of active ingredients and other therapeutic means;
preparing autografts.

Examples of use of the material with eukaryotic cells in vivo are in particular:
implanting a colonized support for tissue (bone, cartilage, dental, gingival) repairs;
coating prostheses (joint prostheses, cardiovascular prostheses);
injecting colonized microbeads for cell vectorization;
applying the support to the skin in the form of a dressing for repairing lesions and/or providing skin treatment.

The use of the material according to the invention in vivo is in particular made possible by virtue of its good mechanical properties, its lack of cytotoxicity and its gradual bioresorption. Indeed, the subcutaneous implantation of the material according to the invention, colonized with mesenchymal stem cells, has been successfully tested in Fischer rats. The support is gradually degraded by the enzymes secreted by the mesenchymal stem cells. The latter colonized the support and entered into a differentiation process. This makes it possible to envision treatments for traumatic cartilage lesions and the prevention of arthroses, by creating a cell substitute for cartilage reconstruction while at the same time preventing connective cicatrization or ossification of the lesions.

The live cells having proliferated on the support can advantageously be recovered intact by means of a mild treatment by contact with a solution of enzymes, for example a mixture of trypsin and collagenase.

It is also possible to envision that the support comprising live cells can be used for cutting thin sections with a microtome, intended for histological, cytological or immunohistochemical analyses, or for cutting ultrafine sections with an ultramicrotome which can be analyzed by observation by transmission electron microscopy.

The invention will now be illustrated by the following nonlimiting examples.

### EXAMPLES

### Figures

FIG. 1a is a scanning electron microscopy (SEM) image of the dense face of the solid support made of PLA prepared in example 1.

FIG. 1b is an SEM image of the section of the solid support made of PLA prepared in example 1.

FIG. 1c is an SEM image at high magnification of the porous face of the solid support made of PLA prepared in example 1.

FIG. 1d is an SEM image of the porous face of the solid support made of PLA prepared in example 1.

FIG. 2a is an SEM image of the microspherical support made of PLA prepared in example 4.

FIG. 2b is an image taken with a video microscope (Zeiss 200M, France) of the microspherical support of example 4 after 7 days of culture with fluorescent HMEC-1 cells.

FIG. 3 represents the change in the contact angle of water with the solid support of PLA of example 5 as a function of the HA deposit time at pH 2 and at pH 6.

FIG. 4 represents the optical density of a double-porosity PLA support (PLA), of a double-porosity PLA support positively charged by aminolysis reaction (aminolyzed PLA), of a double-porosity PLA support positively charged by aminolysis reaction and then functionalized with HA (HA-functionalized PLA) and of a negative control (NC), each support being placed in culture with human mesenchymal stem cells according to example 6.

FIG. 5 is an optical microscope image of the porous face of the solid support made of poly 3HB-co-4HB prepared in example 8.

FIG. 6 is an optical microscope image of the porous face of the solid support made of PCL prepared in example 9.

FIG. 7 is an optical microscope image of the porous face of the solid support made of PBSA prepared in example 10.

FIG. 8 is an optical microscope image of the porous face of the solid support made of a mixture of PLA and PBSA prepared in example 11.

FIG. 9a is an SEM image of the dense face and of the cross section of the tubular support made of PLA prepared in example 13.

FIG. 9b is an SEM image of a portion of the porous internal face of the tubular support made of PLA prepared in example 13.

FIG. 10 is an image taken with a video microscope (Zeiss 200M, France) at 12 days of co-culture of hMSC (in blue) and of HT-29 (in green) on a solid support made of PLA prepared in example 1 and functionalized with HA having a molar mass equal to 880 g·mol⁻¹ as described in example 14. The scale bar corresponds to 20 μm.

### MEASURING METHODS

The diameter of the pores of the support was measured according to the following technique. Images were taken under optical or electron microscopes with corresponding size scales and analyzed with an imaging processing software (ImageJ).

The void fraction of the support was measured according to the following technique. The dry support sample (approximately 50 mg) is weighed on a scale accurate to 1/10th of a mg. The weight W0 is thus obtained. The sample is then immersed in butan-1-ol for 2 h. Butan-1-ol is absorbed in a negligible amount by the studied polyesters, but it wets them well and fills the volume of the pores. Once the sample is removed from the butan-1-ol it is meticulously wiped with a cellulose paper and then weighed. The weight W1 is thus obtained. The void fraction is obtained according to the equation:

$$P = \frac{\frac{W 1 - W 0}{Db}}{\frac{W 1 - W 0}{Db} + \frac{W 0}{Dp}}$$

wherein Db, the density of the butan-1-ol, is equal to 0.81 g·cm⁻³ and Dp is the density of the support. For PLA, Dp is equal to 1.25 g·cm⁻³.

The contact angle of the water at the surface of the support was measured with a Ramé-Hart goniometer (model 100-00). For each support sample analyzed, three drops of Milli-Q water were deposited on the support sample and the values of the contact angles formed to the left and to the right of each drop were measured. The mean value of the contact angle of the water and also the standard deviation are calculated, on the basis of these data, for each support sample.

The amount of HA immobilized at the surface of the support was measured using a QCM-D quartz crystal microscale (Q-Sense, model D300). The dense PLA film was directly deposited on the QCM-D sensor and then modified by aminolysis with a solution of HDA. After rinsing with Milli-Q water, the film positively charged by aminolysis was brought into contact with an aqueous solution of HA and the frequencies of the sensor were monitored over time. The shifts in frequencies of the quartz sensor were translated into weight variations due to the deposit of molecules by interactions on the dense film by means of the Sauerbrey equation. The surface weight of hydrated HA immobilized at the surface of the support was measured after 120 min of deposit of HA followed by washing with a sodium chloride solution at 0.05

The optical density (O.D.) was measured with the WST-1 (Water Soluble Tetrazolium) cell proliferation assay which is a colorimetric assay based on the conversion, by the viable cells, of a tetrazolium salt into a soluble colored compound: formazan. The WST-1 reagent comes from Roche Applied Sciences (France). At the end of the assay, the samples are placed in the wells of a 96-well plate and their O.D. is measured at 420 nm with a Powerwave X plate reader, Bio-tek instruments (France).

### Example 1

### Preparation of a Solid Support Made of PLA

The PLA was supplied by the company NaturePlast (Caen, France) under the trade reference PLE 005. It was composed of 96% L enantiomer and 4% D enantiomer. Its weight-average molar mass (M_{w}) and its number-average molar mass (Mₙ) were determined by HPLC-SEC-MALLS-RI and were respectively equal to 145×10³ g·mol⁻¹ and 60×10³ g·mol⁻¹.

The PLA granules were dissolved in N,N-dimethylformamide (DMF) at a concentration of 7% by weight with stirring at 70° C. Poly(ethylene glycol) (PEG) having a weight-average molar mass (M_{w}) of 8,000 g·mol⁻¹ was added to the solution of PLA in DMF in order to obtain a PEG concentration in the solution of 7% by weight. The solution thus obtained was deposited on a glass plate and spread so as to form a film using a Gardner knife. The thickness of the film was adjusted by means of blocks so as to obtain more or less thick supports. The coagulation of the support was carried out by immersing the glass plate bearing the film in three successive baths of Milli-Q water (Millipore, resistivity of 18 Ω·cm⁻¹) at ambient temperature (20 to 25° C.) The immersing time was 30 min for the first bath and 15 min for the next two baths.

A solid support made of PLA with variable dimensions according to the width of the knife, the thickness of the blocks and the length of the film spread on the glass plate was obtained. For example, when 10 ml of solution of the polymer were spread on an area of 20 cm×15 cm with a block thickness of 180 μm, a membrane of 20 cm×15 cm having a thickness of 120 μm was obtained.

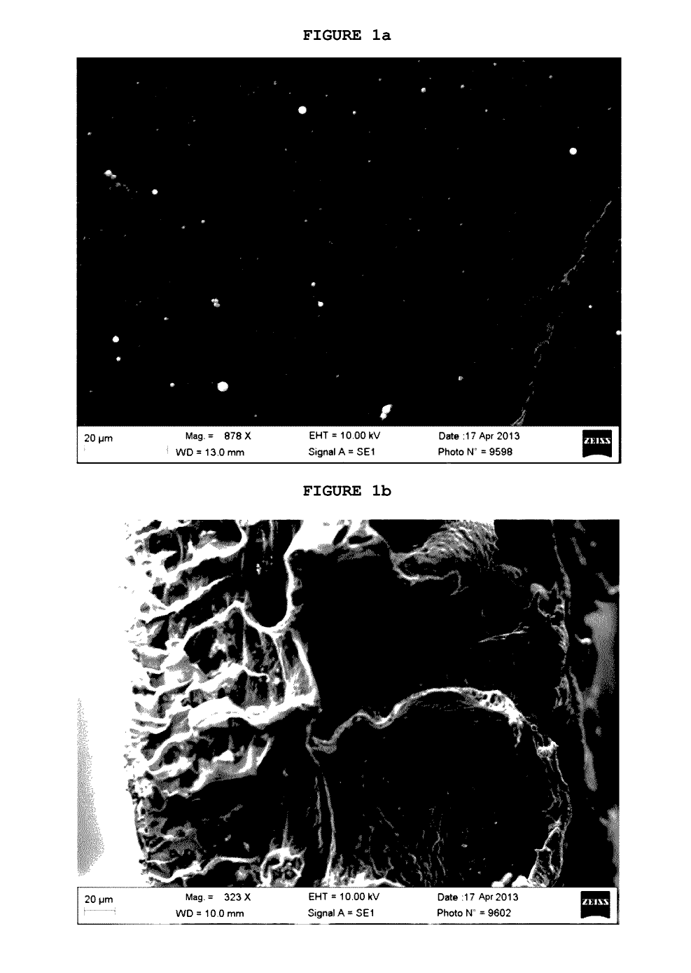

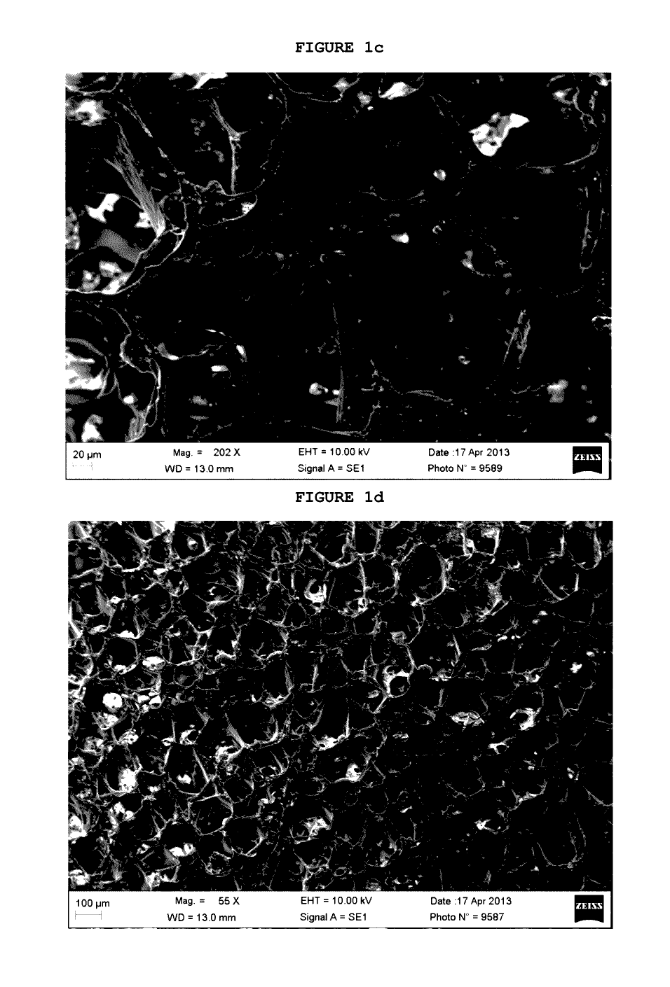

The support had a porous face and a dense face. Images taken on a scanning electron microscope show that the porous face had macropores having a diameter of 160 μm (FIG. 1d) and that the dense face had micropores having a diameter of 1 μm (FIG. 1a). The micropores and the macropores were interconnected, as shown in the images of the section (FIG. 1b) and of the porous face (FIG. 1c) of the support. The void fraction of the support was approximately 75%.

### Example 2

### HA-Functionalization of the Solid Support Made of PLA

The support made of PLA of example 1 was immersed in a solution of 1,6-hexanediamine (HDA) in propan-1-ol at 5 g·l⁻¹ for 30 min at ambient temperature. The support was thoroughly rinsed with Milli-Q water (Millipore, resistivity of 18 Ω·cm⁻¹) in order to remove the propan-1-ol and the unreacted HDA.

The positively charged support was then functionalized with HA having a weight-average molar mass of 3,000,000 g·mol⁻¹ which came from the company HTL Biotechnologies (Javené, France, batch of 01/2009). For that, the support was immersed for 15 minutes in an aqueous solution of HA at 1 g·l⁻¹, the pH of which was adjusted to 2 by adding hydrochloric acid. The pH was measured with a glass electrode (Radiometer Analytical, XC161) connected to a pH-meter (Metrohm, 632). The functionalized support was then rinsed with Milli-Q water in order to remove the unattached HA.

The HA-functionalized supports made of PLA, obtained above, were sterilized either by immersion for a few minutes in pure ethanol or by gamma-irradiation at 32 kGy for 30 min. The sterilized supports were placed in culture wells of 24-well plates. They were then seeded with human microvascular endothelial cells (HMEC-1) in a proportion of 10⁶ cells per support. The proliferation medium was an MCDB base medium supplemented with glutamine (2 mM), fetal calf serum (FCS) decomplemented at 56° C. for 30 min (15%), penicillin (100 IU·ml⁻¹), streptomycin (100 μg·ml⁻¹), hydrocortisone (1 μg·ml⁻¹) and epidermal growth factor (EGF) (10 ng·ml⁻¹).

After 3 days of culture, the proliferation of the HMEC-1 cells on the various materials was quantified using the WST-1 test.

After 3 days of culture, 200 μl of WST-1 reagent were added to each well and, after 4 hours of incubation at 37° C. in the presence of CO₂ (5%), an optical density (O.D.) measurement was carried out on a sample of 70 μl taken from each well according to the method described herein. This O.D. is proportional to the number of live cells. The negative control (NC) corresponded to the proliferation of HMEC-1 in a culture well free of support. The results are given in the table below.

| | Optical density |
|---|---|
| Negative control | 0.571 |
| Material sterilized with ethanol | 1.550 |
| Material sterilized by gamma-irradiation | 1.547 |

### Example 3

### Use of the HA-Functionalized Solid Support In Vivo

The HA-functionalized support made of PLA of example 2 was sterilized with a mixture of ethanol/water at 70% (v/v) and then placed in a culture well. The membrane support was seeded with rat mesenchymal stem cells (rMSC), isolated from the bone marrow of rat femoral head and then transfected with green fluorescent protein (GFP), in a proportion of 500×10³ cells per support. The proliferation medium was composed of the α-MEM base medium supplemented with glutamine (2 mM), fetal calf serum (FCS) decomplemented at 56° C. for 30 min (10%), penicillin (100 IU·ml⁻¹) and streptomycin (100 μg·ml⁻¹). After 2 days of incubation in the proliferation medium, the support was implanted subcutaneously in a Fischer rat.

After 6 weeks of implantation, the PLA support colonized by rMSC was recovered under general anaesthesia (Ketamine/Largactil) with a part of the skin. The sample recovered was directly immersed in paraformaldehyde diluted to 4% for fixing for 48 h. Next, each sample was coated in paraffin and cut using a microtome so as to produce 5 μm-thick serial sections thereof. The sections were then observed under a fluorescence microscope (Leica) in order to reveal the presence of the rMSC-GFP.

A decrease in the diameter of the seeded support of 2.8 cm to 1.2 cm was first of all observed, which reflected a significant degradation process on the support by the enzymes secreted by the rMSC.

The observation under a fluorescence microscope of the 5 μm sections resulting from the recovery of the support implanted in the rat showed that, after 6 weeks of subcutaneous implantation, the rMSC remained viable and adherent to the support. In addition, the cells exhibited a morphology different than that observed before implantation: the cells having a spherical appearance. Furthermore, the cells had grouped together in clumps over the entire depth of the pores.

### Example 4

### Preparation, Functionalization and Culture of Cells on a Microspherical Support Made of PLA

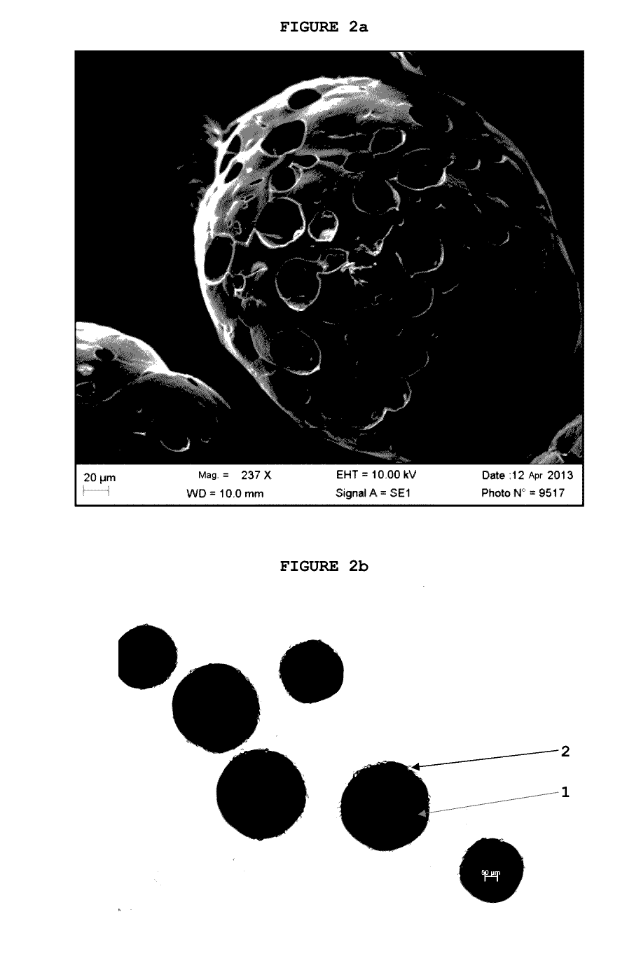

PLA granules (1 g) identical to those of example 1 were dissolved in dichloromethane (20 g) and hexane (4 ml) at a temperature of 40° C. This PLA solution was introduced into 120 ml of an aqueous solution comprising polyvinyl alcohol having a molar mass of 23,000 g·mol⁻¹, at a concentration of 8 g·l⁻¹, with stirring. Everything was kept stirring until complete evaporation of the volatile solvents after 24 hours. The microspherical supports were recovered by filtration and washed with water in order to remove the surfactant. An image taken on a scanning electron microscope shows that the diameter of the microspherical supports was approximately 160 μm, the size of the micropores was approximately 5 μm and the size of the macropores ranged from 20 to 35 μm (FIG. 2a).

These microspherical supports were then functionalized with HA according to the procedure described in example 2.

The functionalized microspherical supports were sterilized with a mixture of ethanol/water at 70% (v/v) and then placed in a culture well of a 24-well plate in a proportion of 0.05 g per well. The microspherical supports were seeded with human microvascular endothelial cells (HMEC-1) sold by ATCC under the reference CRL-3243™ and labeled with a green fluorescent label sold by Life Technologies under the reference CellTracker® Green CMFDA Dye, before being seeded on the microspheres in a proportion of 500×10³ cells per well. The proliferation medium was an MCDB base medium supplemented with glutamine (2 mM), fetal calf serum (FCS) decomplemented at 56° C. for 30 min (15%), penicillin (100 IU·ml¹), streptomycin (100 μg·ml⁻¹), hydrocortisone (1 μg·ml⁻¹) and epidermal growth factor (EGF) (10 ng·ml⁻¹). After 7 days of incubation in the proliferation medium, good adhesion and good proliferation of the HMEC-1 cells on the microspherical supports were observed.

The image taken with a video microscope (FIG. 2b) after seeding with the HMEC-1 cells shows the fluorescent cells (2) in [ deposited at the surface of the microspheres of material according to the invention (1) in black.

### Example 5

### Influence of the pH of the Solution of Bioactive Molecules on the Contact Angle of the Water

This example makes it possible to study the influence of the pH on the efficiency of deposit of HA of high molar mass during the step of functionalizing the support. For reasons of analysis simplicity, the experiments were carried out on dense PLA supports and not on double-porosity PLA supports such as those of the present invention. Nevertheless, the results obtained on the dense PLA supports are transposable to the double-porosity PLA supports.

The dense PLA support was prepared by means of a solvent evaporation process. The PLA used was identical to that of example 1. PLA solutions were prepared by dissolving the PLA granules in chloroform at approximately 35-40° C., with magnetic stirring, for 4 to 5 hours. The support was prepared with a solution of PLA at 6.66% (w/w), obtained by dissolving 1 g of PLA in 15 g of chloroform. After dissolution of the PLA, the solution was poured into a glass Petri dish, immediately covered in order to prevent excessively fast evaporation of the chloroform. The dense PLA support was finally obtained after evaporation of the solvent for 6 to 7 days at ambient temperature. The support had a thickness of about 120 μm and was stored in a desiccator at ambient temperature.

The aminolysis reaction was carried out by immersing the PLA support in a solution of HDA at 10 g·l⁻¹ in propan-1-ol for 60 min. The support was then thoroughly rinsed with Milli-Q water (Millipore, resistivity of 18 Ω·cm⁻¹) so as to remove the unreacted HDA and the propan-1-ol. After aminolysis, the support was stored in a desiccator at ambient temperature.

The positively charged support was then functionalized with an HA having a weight-average molar mass of 3,000,000 g·mol⁻¹ identical to that used in example 2. For that, the support was immersed in an aqueous solution of HA at 1 g·l⁻¹, the pH of which was adjusted to 2 or 6 by adding hydrochloric acid or potassium hydroxide. The pH was measured with a glass electrode (Radiometer Analytical, XC161) connected to a pH-meter (Metrohm, 632). The functionalized support was then rinsed with Milli-Q water in order to remove the HA that had not attached. The functionalized support was stored in a desiccator at ambient temperature.

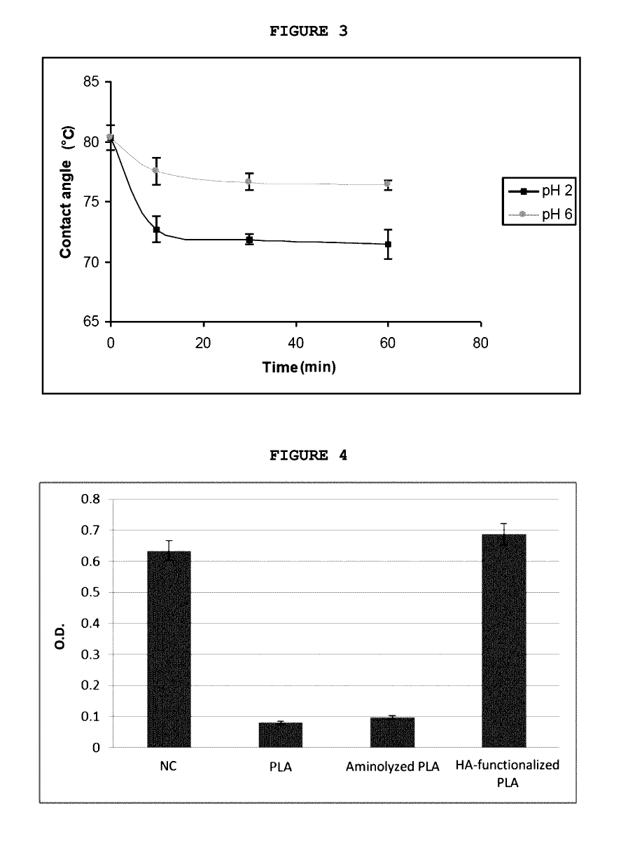

For each support, the contact angle of the water at the surface of the functionalized support was measured according to the method described herein. FIG. 3 shows a decrease in the value of the contact angle of the water with a dense PLA solid support that is greater when the HA solution is at pH 2 than when it is at pH 6. This reflects a greater increase in the hydrophilic nature of the surface of the support during the depositing at pH 2. Thus, the depositing of HA occurs much more successfully at pH 2 than at pH 6.

Similar results were obtained with HA having a weight-average molar mass of 350,000 g·mol⁻¹.

On the other hand, with HA having a weight-average molar mass of 880 g·mol⁻¹, the depositing of HA occurs more successfully at pH 6 than at pH 2.

### Example 6

### Influence of the pH of the Solution of Bioactive Molecules on the Amount of Bioactive Molecules Deposited

This example also makes it possible to study the influence of the pH on the efficiency of the depositing of HA of high molar mass during the support functionalization step. For reasons of analysis simplicity, the experiments were carried out on dense PLA supports and not on double-porosity PLA supports such as those of the present invention. Nevertheless, the results obtained on the dense PLA supports are transposable to the double-porosity PLA supports.

The dense PLA support was prepared by spin-coating directly on a quartz crystal QCM-D sensor. For that, a uniform dense film was deposited using a 1% (w/w) solution of PLA in ethyl acetate and then the solvent was evaporated under vigorous rotation of the quartz crystal QCM-D sensor. The resulting support was then positively charged by aminolysis and then functionalized with HA at 2 different pH values as described in example 5, with, for each test, the HDA concentration, the aminolysis time, the weight-average molar mass of HA and the HA concentration being replaced with the respective values given in table 1 below. For each test, the amount of HA immobilized at the surface of the support was measured according to the method described herein.

TABLE 1

| | | HA deposit | | | |
|---|---|---|---|---|---|
| | | | | Weight per unit of | |
| Aminolysis | | | | surface area of HA | |
| [HDA] | Duration | M_{w} of HA | [HA] | (ng · cm⁻²) | |
| (g · l⁻¹) | (min) | (g · mol⁻¹) | (g · l⁻¹) | at pH 2 | at pH 6 |
| 5 | 10 | 3 × 10⁶ | 0.1 | 1150 | 240 |
| 5 | 60 | 3 × 10⁶ | 0.1 | 560 | 160 |
| 5 | 10 | 880 | 1 | 240 | 780 |

For the support functionalized with HA having a weight-average molar mass of 3,000,000 g·mol⁻¹, the weight per unit of surface area of hydrated HA immobilized at the surface of the PLA support was always much higher at pH 2 than at pH 6.

On the other hand, with HA having a weight-average molar mass of 880 g·mol⁻¹, the weight per unit of surface area of hydrated HA immobilized at the surface of the PLA support was higher at pH 6 than at pH 2.

### Example 7

### Influence of the HA-Functionalization of the Porous Supports on Cell Proliferation

A double-porosity PLA support as prepared in example 1, a PLA support modified by aminolysis as described in example 2 and a PLA support modified by aminolysis and then functionalized with HA as described in example 2 were sterilized with a mixture of ethanol/water at 70% (v/v) and then placed in culture wells of a 24-well plate. The supports were seeded with human mesenchymal stem cells (hMSC), sold by ATCC under the reference PCS-500-010™, in a proportion of 250×10³ cells per support. The proliferation medium was composed of the α-MEM base medium supplemented with glutamine (2 mM), fetal calf serum (FCS) decomplemented at 56° C. for 30 min (10%), penicillin (100 IU·ml⁻¹) and streptomycin (100 μg·ml⁻¹). The hMSC were cultured for 7 days at 37° C. in the presence of CO₂ (5%). After 7 days of culture, 200 μl of WST-1 reagent were added to each well and, after 4 hours of incubation at 37° C. in the presence of CO₂ (5%), an optical density (O.D.) measurement was carried out on a sample of 70 μl taken from each well according to the method described herein. This O.D. is proportional to the number of live cells. The negative control (NC) corresponded to the proliferation of hMSC in a culture well free of support.

It is observed in FIG. 4 that there was indeed immobilization of HA on the porous supports since the hMSC proliferation was much higher when the porous supports had been functionalized with HA. This shows that, with porous supports, there was indeed aminolysis and then immobilization of HA as far as the inside of the pores.

### Example 8

### Preparation and Functionalization of a Solid Support Made of Poly 3HB-Co-4HB

Solid supports made of 3-hydroxybutyrate/4-hydroxybutyrate copolymer (poly 3HB-co-4HB) were prepared according to the process described in example 1, starting from 3HB-co-4HB supplied by Tianjin GreenBio Materials (China), having a weight-average molar mass (M_{w}) of 700,000 g·mol⁻¹ and an average 4-hydroxybutyrate content of 12 mol %.

The poly 3HB-co-4HB granules were dissolved in N-methyl-2-pyrrolidone (NMP) at a concentration of 8.3% by weight with stirring at 80° C. PEG having a weight-average molar mass (M_{w}) of 8,000 g·mol⁻¹ was added to the solution of poly 3HB-co-4HB in NMP so as to obtain a PEG concentration in the solution of 8.3% by weight.

The subsequent steps for achieving the support, comprising in particular the depositing of the polymer on a glass plate and the coagulation of the support by immersion in several successive baths of Milli-Q water, were carried out as described in example 1.

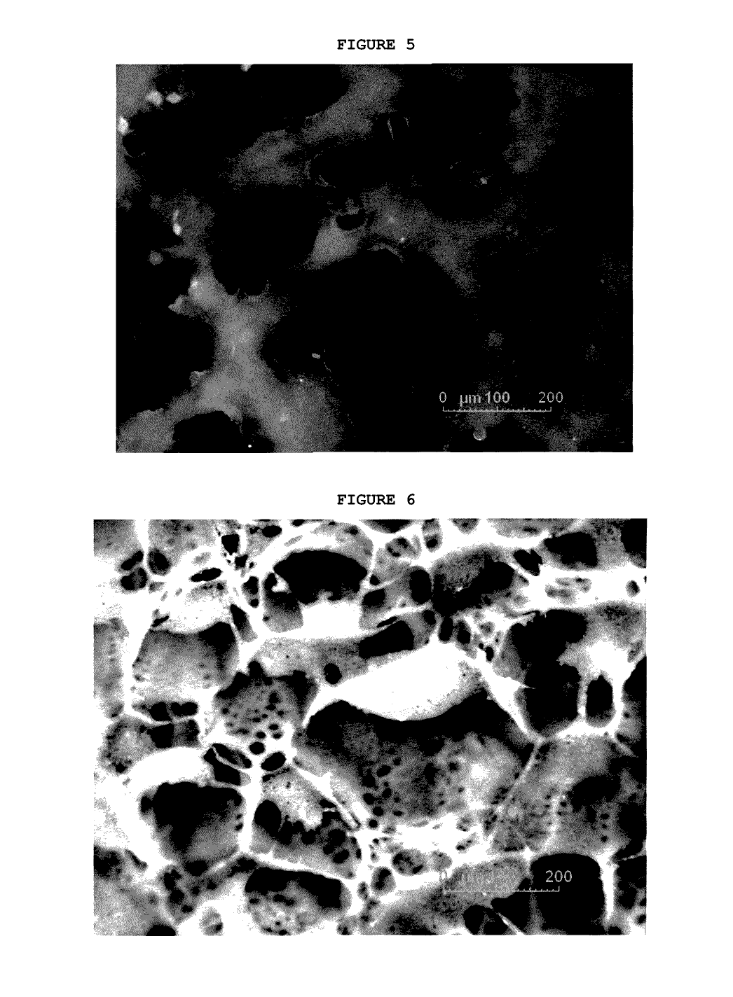

The support had a porous face and a dense face. An image taken with an optical microscope shows that the porous face had macropores having a diameter of approximately 110 μm and micropores having a diameter of approximately 2 μm (FIG. 5). The void fraction of the support was approximately 65%.

The steps of aminolysis and of functionalization of the positively charged support with HA were carried out in accordance with example 2.

### Example 9

### Preparation and Functionalization of a Solid Support Made of PCL

Solid supports made of poly(caprolactone) (PCL) were prepared according to the process described in example 1, starting from PCL supplied by Sigma Aldrich having an average molar mass Mₙ=80,000 g·mol⁻¹.

The PCL granules were dissolved in DMF at a concentration of 8.3% by weight with stirring at 80° C. Polyvinylpyrrolidone (PVP) having a weight-average molar mass (M_{w}) of 30,000 g·mol⁻¹ was added to the solution of PCL in DMF so as to obtain a PVP concentration in the solution of 1.8% by weight.

The subsequent steps for achieving the support, comprising in particular the depositing of the polymer on a glass plate and the coagulation of the support by immersion in several successive baths of Milli-Q water, were carried out as described in example 1.

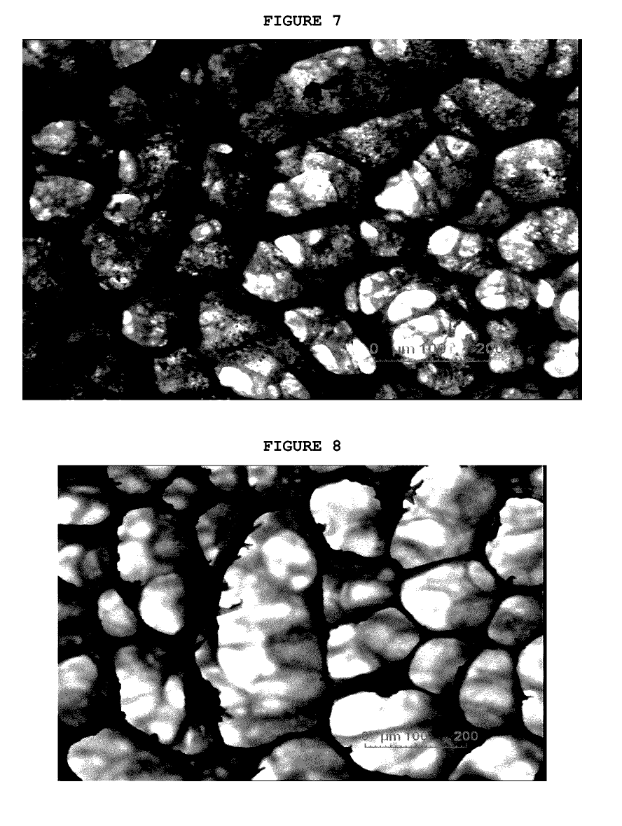

The support had a porous face and a dense face. An image taken with an optical microscope shows that the porous face had macropores having a diameter of approximately 120 μm and micropores having a diameter of approximately 2 μm (FIG. 6). The void fraction of the support was approximately 75%.

The steps of aminolysis and of functionalization of the positively charged support with HA were carried out in accordance with example 2.

### Example 10

### Preparation and Functionalization of a Solid Support Made of PBSA

Solid supports made of butylene succinate/butylene adipate (PBSA) copolymer were prepared according to the process described in example 1, starting from PBSA supplied by NaturePlast under the reference 3PBE 001, having an average molar mass M_{w}=165,000 g·mol⁻¹.

The PBSA granules were dissolved in DMF at a concentration of 7% by weight with stirring at 80° C. PEG having a weight-average molar mass (M_{w}) of 8,000 g·mol⁻¹ was added to the solution of PBSA in DMF so as to obtain a PEG concentration in the solution of 7% by weight.

The subsequent steps for achieving the support, comprising in particular the depositing of the polymer on a glass plate and the coagulation of the support by immersion in several successive baths of Milli-Q water, were carried out as described in example 1.

The support had a porous face and a dense face. An image taken with an optical microscope shows that the porous face had macropores having a diameter of approximately 130 μm and micropores having a diameter of approximately 10 μm (FIG. 7). The void fraction of the support was approximately 75%.

The steps of aminolysis and of functionalization of the positively charged support with HA were carried out in accordance with example 2.

### Example 11

### Preparation and Functionalization of a Solid Support Comprising a Mixture of PLA and PBSA

Solid supports made of a mixture of PLA and PBSA were prepared according to the process described in example 1, starting from the PLA described in example 1 and from the PBSA described in example 10.

The PLA granules and PBSA granules were dissolved in DMF with stirring at 80° C. at a concentration of 2.1% by weight for the PLA and 4.9% by weight for the PBSA. PEG having a weight-average molar mass (M_{w}) of 8,000 g·mol⁻¹ was added to the solution of PLA and PBSA in DMF so as to obtain a PEG concentration in the solution of 7% by weight.

The subsequent steps for achieving the support, comprising in particular the depositing of the polymer on a glass plate and the coagulation of the support by immersion in several successive baths of Milli-Q water, were carried out as described in example 1.

The support had a porous face and a dense face. An image taken with an optical microscope shows that the porous face had macropores having a diameter of approximately 150 μm and micropores having a diameter of approximately 10 μm (FIG. 8). The void fraction of the support was approximately 75%.

The steps of aminolysis and of functionalization of the positively charged support with HA were carried out in accordance with example 2.

### Example 12

### Attaching of Human Microvascular Endothelial Cells to the Materials of Examples 8 to 11

The HA-functionalized supports prepared in examples 8 to 11 were sterilized with a mixture of ethanol/water at 70% (v/v) and then placed in the culture wells of a 24-well plate. The membrane supports were seeded with human microvascular endothelial cells (HMEC-1), sold by ATCC under the reference CRL-3243™, in a proportion of 10⁶ cells per support. The proliferation medium was an MCDB base medium supplemented with glutamine (2 mM), fetal calf serum (FCS) decomplemented at 56° C. for 30 min (15%), penicillin (100 IU·ml⁻¹), streptomycin (100 μg·ml⁻¹), hydrocortisone (1 μg·ml⁻¹) and epidermal growth factor (EGF) (10 ng·ml⁻¹).

After 3 days of culture, 200 μl of WST-1 reagent were added to each well and, after 4 hours of incubation at 37° C. in the presence of CO₂ (5%), an optical density (O.D.) measurement according to the method described herein was carried out on a sample of 70 μl taken from each well. This O.D. was proportional to the number of live cells. The negative control (NC) corresponded to the proliferation of HMEC-1 in a culture well free of support (plastic).

| | Negative | HA-functionalized |
|---|---|---|
| Optical density | control | support |
| poly 3HB-co-4HB | 0.114 | 1.157 |
| (example 8) | | |
| PCL (example 9) | 0.041 | 1.341 |
| PBSA | 0.140 | 1.214 |
| (example 10) | | |
| PLA-PBSA | 0.139 | 1.264 |
| (example 11) | | |

Good adhesion and good proliferation of the HMEC-1 cells on the materials prepared according to examples 8 to 11 were observed.

### Example 13

### Preparation and Functionalization of a Hollow Support Made of PLA

Hollow supports of cylindrical shape were prepared according to the protocol described in example 1, with the flat substrate for the deposit of the polymer film being replaced with a cylindrical tube.

The solution of the polymer which comprises PLA at 7% by weight in DMF was prepared by dissolving PLA granules in N,N-dimethylformamide (DMF) at a concentration of 7% by weight with stirring at 70° C.

The solution of the polymer was deposited on a Teflon cylindrical tube 1.5 mm in external diameter and 5 cm in length, by dipping said tube in said solution of the polymer.

The coagulation of the support was carried out by immersion of the plastic cylindrical tube bearing the film in three successive baths of Milli-Q water (Millipore, resistivity of 18 Ω·cm⁻¹) at ambient temperature (20 to 25° C.). The immersion time was 30 min for the first bath and 15 min for the next two baths.

A hollow support made of PLA, with variable dimensions according to the diameter and the length of the plastic cylinder used and the duration of immersion of the cylinder in the solution of the polymer, was obtained. In this example, the tubular support obtained had a thickness of 180 μm. Such tubular supports have a porous face on the inside, and an architecture in the thickness similar to that of the flat supports.

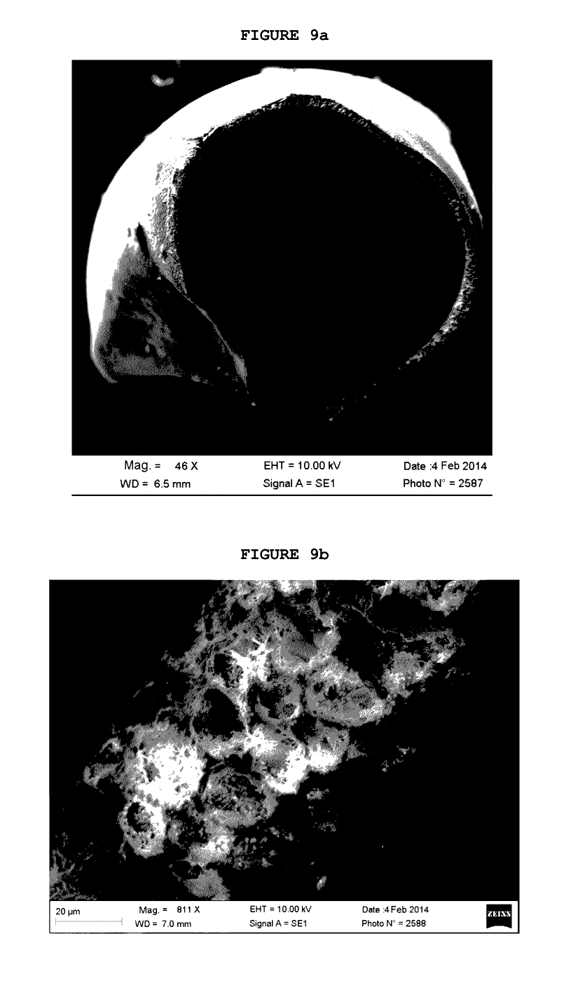

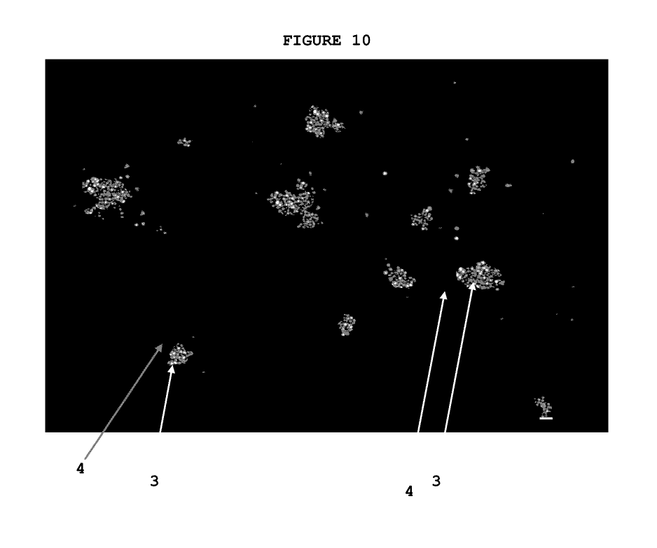

Images taken with a scanning electron microscope showed that the cross section of the support is tubular (FIG. 9a) and that the support had a double porosity (FIG. 9b). The macropores had a diameter of approximately 20 μm, the micropores had a diameter of approximately 1 μm and the void fraction of the support was approximately 60%.

### Example 14

### Co-Culture of hMSC and of Colon Cancer Cells on PLA Supports Functionalized with HA of Low Molar Mass

The porous supports of PLA were prepared as described in example 1 and then functionalized according to the following protocol:

The PLA support of example 1 was immersed in a solution of 1,6-hexanediamine (HDA) in propan-1-ol at 5 g·l⁻¹ for 15 min at ambient temperature. The support was thoroughly rinsed with Milli-Q water (Millipore, resistivity of 18 Ω·cm⁻¹) in order to remove the propan-1-ol and the unreacted HDA.

The positively charged support was then functionalized with HA having a weight-average molar mass of 880 g·mol⁻¹, obtained in the laboratory by catalyzed hydrolysis, with hyaluronidase, of the HA of high molar mass (M_{w}=3,000,000 g·mol⁻¹, HTL Biotechnologies (Javené, France, batch of 01/2009)). The support was immersed for 15 minutes in an aqueous solution of HA at 1 g·l⁻¹, the pH of which was adjusted to 6 by adding hydrochloric acid. The pH was measured with a glass electrode (Radiometer Analytical, XC161) connected to a pH-meter (Metrohm, 632). The functionalized support was then rinsed with Milli-Q water in order to remove the HA that had not attached.

The membrane support, after having been sterilized with a mixture of ethanol/water at 70% (v/v), was seeded with human mesenchymal stem cells (hMSC) and colon cancer endothelial cells (HT-29) in a total proportion of 10⁶ cells per support. The cell ratio was 2:1 for hMSC: HT-29. The hMSC (reference PCS-500-010™) and the HT-29 (reference HTB-38™) come from ATCC. The hMSC were labeled beforehand with a blue fluorescent dye, Cell Tracker™ Blue CMAC (Life technologies, France), and the HT-29 were labeled with a green fluorescent dye, Cell Tracker™ Green CMFDA (Life technologies, France). The proliferation medium was composed of the α-MEM base medium supplemented with glutamine (2 mM), fetal calf serum (FCS) decomplemented at 56° C. for 30 min (10%), penicillin (100 IU·ml⁻¹) and streptomycin (100 μg·ml⁻¹).

After 12 days of culture, it was observed, on the image obtained with a video microscope (Zeiss 200M, France) of FIG. 10, that the hMSC in blue (4) had come to surround the voluminous spheroids of HT-29 in green (3). The obtention of spheroids showed the three-dimensional organization of the cells in the material. Furthermore, the organization of the hMSC with respect to the HT-29 showed that there was communication between the two cell types within the material.

1-15. (canceled)

16. A three-dimensional material with double-porosity comprising a support constituted of a polymer of which the surface comprises positive charges and is functionalized with bioactive molecules which comprise negative charges and are suitable for increasing eukaryotic cell proliferation.

17. The material as claimed in claim 16, wherein the polymer is chosen from polyesters.

18. The material as claimed in claim 17, wherein the polymer is a bioresorbable polyester chosen from homopolymers and copolymers of hydroxy acids; polycaprolactone; homopolymers and copolymers of poly(butylene succinate) and of poly(butylene adipate); and mixtures thereof.

19. The material as claimed in claim 18, wherein the bioresorbable polyester is polylactic acid.

20. The material as claimed in claim 16, wherein the bioactive molecules are chosen from polysaccharides; proteins; peptides; and mixtures thereof.

21. The material as claimed in claim 20, wherein the bioactive molecules are hyaluronan.

22. The material as claimed in claim 16, wherein the material comprises interconnected micropores and macropores, the micropores having a diameter of less than 20 μm, and the macropores having a diameter of from 20 to 199 μm.

23. The material as claimed in claim 22, wherein the micropores have a diameter of from 0.1 to 10 μm.

24. The material as claimed in claim 22, wherein the macropores have a diameter of from 20 to 199 μm.

25. The material as claimed in claim 16, wherein the support has a solid, hollow or microspherical shape, preferably solid shape.

26. The material as claimed in claim 25, wherein the support has a solid shape.

27. A process for producing a three-dimensional material, which comprises the following successive steps:
forming a polymer by phase inversion so as to obtain a two- or three-dimensional support with double-porosity,
treating the surface of the support so as to confer positive charges thereon, and
functionalizing the thus-treated support with bioactive molecules.

28. The process as claimed in claim 27, wherein the step of forming the polymer by phase inversion comprises the steps of:
preparing a solution of the polymer comprising said polymer, at least one solvent for the polymer and optionally at least one pore-forming agent and/or at least one organic or inorganic compound; and
introducing said solution of the polymer into an aqueous solution optionally comprising at least one other water-miscible solvent and/or at least one surfactant.

29. The process as claimed in claim 28, wherein said solution of the polymer comprises the polymer, at least one solvent for the polymer which is water-miscible, optionally at least one pore-forming agent and optionally at least one organic or inorganic compound, wherein said solution of the polymer is deposited on a solid or hollow substrate and wherein the phase inversion is carried out by immersing said substrate in an aqueous solution optionally comprising at least one other water-miscible solvent, so as to obtain, respectively, a solid or hollow support.

30. The process as claimed in claim 28, wherein said solution of the polymer comprises the polymer, at least one volatile solvent with low water-miscibility and optionally at least one organic or inorganic compound, and wherein the phase inversion is carried out by introducing said solution of the polymer into an aqueous solution comprising at least one surfactant, followed by evaporation of said volatile solvent, so as to obtain a microspherical support.

31. The process as claimed in claim 28, wherein the surface treatment is carried out by aminolysis reaction of the polymer using a solution of at least one aliphatic α,ω-diamine.

32. The process as claimed in claim 28, wherein the functionalizing step is carried out by immersing the support in a solution of bioactive molecules, followed by rinsing.

33. The process as claimed in claim 17, wherein the bioactive molecules are molecules of hyaluronan having a weight-average molar mass of from 20,000 to 10,000,000 g·mol⁻¹ and wherein the pH of the hyaluronan solution is from 1 to 2.9.

34. A method for attaching and stimulating the proliferation of eukaryotic cells comprising attaching eukaryotic cells to the material of claim 16 and stimulating their proliferation.

35. The method as claimed in claim 34, wherein the eukaryotic cells are chosen from stem cells, fibroblasts, endothelial cells, cancer cells, and mixtures thereof.
